(19) United States
(12) Patent Application Publication
(10) Pub. No.: US 2014/0357933 A1
(43) Pub. Date: Dec. 4, 2014

(54) MICROSCOPIC MAGNETIC STIMULATION OF NEURAL TISSUE

(71) Applicant: THE GENERAL HOSPITAL CORPORATION, Boston, MA (US)

(72) Inventors: Seungwoo Lee, Boston, MA (US); Giorgio Bonmassar, Lexington, MA (US); Shelley Fried, Boston, MA (US)

(21) Appl. No.: 14/294,891

(22) Filed: Jun. 3, 2014

Related U.S. Application Data
(60) Provisional application No. 61/830,379, filed on Jun. 3, 2013.

(51) Int. Cl. A61N 2/02

(52) U.S. Cl. CPC A61N 2/02
USPC 600/12

(57) ABSTRACT

An implantable neural stimulation device includes a magnetic coil specifically dimensioned to be implantable inside the tissue and structured to generate, in the vicinity of the target tissue adjacent to which such coils is disposed in operation, magnetic field the strength of which is substantially the same as the strength of magnetic field generated in such tissue during the conventional TMS procedure. The modulation of orientation of microcoil modulates the activation of targeted neuronal tissue.

### CROSS REFERENCE TO RELATED APPLICATIONS

This application claims the benefit of U.S. Provisional Patent Application No. 61/830,379 filed on Jun. 3, 2013 and titled “Microscopic Magnetic Stimulation of Neural Tissue”, the entire contents of which are hereby incorporated by reference herein, for all purposes.

### BACKGROUND

Electrical stimulation is currently used to treat a wide range of cardiovascular, sensory, and neurological diseases. Despite its success, there are significant limitations to its application, including incompatibility with magnetic resonance imaging, limited control of electric fields, and decreased performance associated with tissue inflammation. Magnetic stimulation overcomes these limitations but existing devices (that is, those used for transcranial magnetic stimulation) are large, which reduced their applicability to chronic applications. In addition, existing devices are not effective for stimulation of tissue that is located deeper (such as sub-cortical tissue, for example, or for intra-ocular retinal stimulation.

### SUMMARY

Embodiments of the invention provide a method for stimulating a target tissue with a microcoil that has been disposed within the tissue. For example, the target tissue may be sub-cortical tissue (in which case the microcoil may be a subcortical microcoil) or an intra-ocular retinal tissue (and the coil disposed intra-ocularly becomes a retinal microcoil). The method includes applying an electrical pulse to terminals of an implanted microcoil positioned in the vicinity of said target deep tissue to generate a first magnetic field at said deep tissue such that the first magnetic field has substantially the same strength at the target tissue as a strength of a second magnetic field, wherein the second magnetic field is defined in the target tissue during a transcranial magnetic stimulation procedure. The implantable microcoil has dimensions on the order of a millimeter, which, for the purposes of this invention, is defined as dimensions ranging from sub-millimeter dimensions (for example, of about 100 microns or even less) to about 1 . . . 2 millimeters or so. The method also includes eliciting a response of the target tissue with the first magnetic field, wherein the response has latency, and modulating a response of the target tissue by defining a spatial orientation of the implanted microcoil with respect to a surface of the tissue.

The method additionally includes positioning of the microcoil implant at a sub-millimeter distance from the surface of the target tissue and/or eliciting a response, from the target tissue, to light illuminating the tissue. In a related embodiment, the method may include reducing the latency by increasing amplitude of the electric pulse. Eliciting a response with the first magnetic field may, in a specific case, include at least one of (i) a direct activation of a retinal ganglion cell with the first magnetic field and (ii) an indirect activation of the retinal ganglion cell resulting from activation of neurons presynaptic to the retinal ganglion cell. Alternatively or in addition, eliciting a response with the first magnetic field may, in a specific case, include at least one of (i) a direct activation of a cell of a sub-cortical tissue with the first magnetic field and (ii) an indirect activation of such cell resulting from activation of neurons presynaptic to such subcortical cell. Alternatively or in addition, the eliciting a response with the first magnetic field may include eliciting a response of the target subcortical tissue during a procedure of magnetic resonance imaging (MRI) of the target subcortical tissue.

Embodiments additionally provide a tissue stimulator system. Such system contains a biocompatible unit including a implantable coil that is structured (i) to be either subcortically or intra-ocularly disposed in vicinity of a target tissue and (ii) to generate a first magnetic field in a target tissue in response to an electrical impulse applied to the coil. In that, the first magnetic field has substantially the same strength as a second magnetic field, wherein the second magnetic field is defined in the target tissue during a transcranial magnetic stimulation procedure. The system further includes a stimulator operably coupled to the biocompatible unit and containing a power drive providing an electric stimulus (such as a pulse or a different waveform, including but not limited to a sinusoidal waveform or trapezoidal waveform) to the implanted coil; and a processor configured to govern parameters of said electrical stimulus. The processor may be programmed to change, in operation of said system, latency of response of the target tissue to the electrical stimulus. The coil implant of the embodiment has dimensions on the order of a millimeter, ranging from sub-millimeter dimensions up to about 1 . . . 2 mm or so, and is disposed, in operation, at a sub-millimeter distance from a surface of the target tissue. In a specific embodiment, the coil is disposed in association with the biocompatible unit such as to elicit, in operation of the system, a response from the target retinal tissue that includes at least one of (i) a direct activation of a retinal ganglion cell with said first magnetic field and (ii) an indirect activation of the retinal ganglion cell resulting from activation of retinal neurons presynaptic to the retinal ganglion cell. In another specific embodiment, the coil is disposed in association with the biocompatible unit such as to elicit, in operation of the system, a response from the target sub-cortical tissue that includes at least one of (i) a direct activation of a sub-cortical tissue cell with said first magnetic field and (ii) an indirect activation of such cell resulting from activation of neurons presynaptic to this cell.

### BRIEF DESCRIPTION OF THE DRAWINGS

The invention will be more fully understood by referring to the following Detailed Description of Specific Embodiments in conjunction with the generally not-to-scale Drawings, of which:

FIG. 1A is a contour plot illustrating three-dimensional distribution of the electric field around a microcoil starting at about 100 microns from the edge of the coil;

FIG. 1B is a contour plot illustrating three-dimensional distribution of the electric field starting at about 100 microns below the terminal of the coil of FIG. 1A;

FIG. 1C is a plot of the axial distribution of the electric field as a function of radial distance from the coil presented at different elevations along the axis of the coil (from 0 microns to 500 microns, as shown in the legend);

FIG. 1D is a plot of the axial distribution of the electric field as a function of axial position along the coil of FIG. 1A (i.e., an axial distance from the terminal of the coil) at different radiant distances (ranging from 50 microns to 400 microns as shown in the legend);

FIGS. 2A and 2B are diagrams illustrating the structure of an embodiment of the inductor. FIG. 2A is an image of the inductor; FIG. 2B is an image of the inductor the outer layer of which has been chemically dissolved to exposure the structure of the underlying solenoid;

FIGS. 3A and 3B are diagrams illustrating two different orientations of a microcoil of the invention tested with the microcoil axis being parallel (FIG. 3A) and perpendicular (FIG. 3B) to the surface of the target tissue (as shown—retinal tissue);

FIG. 3C is a schematic illustrating an experimental set-up depicting the stimulation of the tissue with a microcoil of the invention and recordation of the activated responses with a cell-attached patch-clamp electrodes positioned, in this specific example, on the surface of soma of retinal ganglion cells. The retinal tissue was illuminated with an IR light and observed with a digital camera;

FIGS. 4A, 4B, 4C, 4D, 4E, and 4F provide experimentally acquired data illustrating that the change of the orientation of the microcoil used for modulation of activation of the target tissue (in this specific case, as shown—retinal tissue) with micro-magnetic stimulation according to an embodiment of the invention affects neural response. FIG. 4A shows response of retinal ganglion cells to a single pulse (shown as “microcoil input”) of micro-MS. FIG. 4B shows plots representing an overlay of light-evoked action potential and micro-MS-evoked biphasic waveform (each averaged over five responses). Horizontal off-set between the plots is added to facilitate clear comparison. FIG. 4C is a peri-stimulus histogram (with a bin-width of 10 ms) of the firing rate for the five repetitions of FIG. 4A. FIG. 4D is a plot of responses (averaged over five traces) of the retinal ganglion cells to a single micro-MS pulse acquired when the main axis of coil was oriented parallel to and 300 microns from the surface of the retinal tissue at hand. FIG. 4E shows a magnified portion “A” of the plot of FIG. 4D. The “spikes” indicate elicited action potentials. FIG. 4F shows one of the five traces of FIGS. 4D, 4E for which an action potential was elicited;

FIG. 5A shows plots illustrating retinal ganglion cell responses a, b, c, and d to a stimulus S provided by the microcoil of the invention, for different strengths of the stimulus (4V, 5V, 5.5V, and 6V, respectively). The microcoil was oriented with its axis parallel to the surface of the retinal tissue at hand;

FIG. 5B shows a summary of cell responses to stimulation (with increasing amplitude) by a microcoil of the invention (for a parallel orientation between the microcoil axis and the surface of the retinal tissue at hand);

FIG. 5C shows a summary of cell responses to stimulation (with increasing amplitude) by a microcoil of the invention (for a perpendicular orientation between the microcoil axis and the surface of the retinal tissue at hand);

FIGS. 5D and 5E are plots illustrating the onset latencies of elicited spikes of responses as a function of amplitude of the stimulating field, for stimulation with a microcoil of the invention when the microcoil axis is parallel (FIG. 5D) and perpendicular (FIG. 5E) to the surface of the retinal tissue. Bars represent standard errors (n=5);

FIGS. 5F and 5G illustrate the sensitivity of neural response to location of the stimulating microcoil of the invention. Number of spikes (averaged over five traces) is plotted as a function of stimulus amplitude for three different separation between the coil and the retinal tissue at hand for parallel (FIG. 5F) and perpendicular (FIG. 5G) orientation between the microcoil axis and the surface of the retinal tissue;

FIG. 6 is a table listing nomenclature, dimensions, and constants used in finite-element method simulations;

FIG. 7A shows a distribution of magnetic field (measured in Tesla) in the yz-plane (x=0) in a coordinate system of FIGS. 1A, 1B;

FIG. 7B is a colormap representing the magnitude of the electric field (Vm⁻¹) induced in and around the microcoil of the invention. The colormap shows the current density at each point of the yz-plane, and the lines uniformly sample the magnetic flux density in 20 bins;

FIG. 8A is a diagram schematically presenting a retinal stimulation system containing a microcoil according to an embodiment of the invention;

FIG. 8B is a diagram of an embodiment of a microcoil with indicators of generated fields.

### DETAILED DESCRIPTION

In accord with preferred embodiments of the present invention, methods and apparatus are disclosed for activation of target neuronal tissue with the use of magnetic coil(s) specifically configured and dimensioned for being disposed inside and adjacent to such target tissue (for example, sub-cortically, or intra-ocularly) and modulation of such activation and/or eliciting specific neuronal responses by varying spatial orientation(s) of the coils relative to the target tissue can be used to generate specific neuronal responses.

Electrical stimulation of excitable tissue is a rapidly expanding viable therapeutic strategy for treating human disorders. For example, deep brain stimulation (DBS) has been successful in the treatment of movement disorders such as Parkinson's disease, dystonia and essential tremor, and clinical trials are currently underway to examine its efficacy for the treatment of additional neurological and psychiatric diseases including epilepsy, major depression and obsessive-compulsive disorder. Electrical stimulation of the muscle has a long therapeutic history; the most notable example is the use of cardiac pacemakers for the treatment of conduction and arrhythmia disorders of the heart. There has also been success in using cochlear implants to restore auditory function, and considerable efforts are underway to develop limb and visual prostheses. Despite the successes of direct electrical stimulation, its implementation comes with some technical and biological limitations. For example, magnetic resonance imaging (MRI) examination of DBS patients can result in neurological damage because of excessive heating at the stimulating electrode tip. In such cases, heating is induced by MRI-generated radio-frequency waves that interact with the conductive leads to generate induced currents (known as the ‘antenna effect’), which result in the loss of energy in the form of heat. In addition, safety concerns have been raised for pacemakers owing to the reported changes in cardiac pacing after MRI that may also be related to contact tip heating. Another challenge with direct electrical stimulation is that the therapeutic effects can be altered by inflammatory and immune reactions of the tissue in response to direct contact with the stimulating electrode. For example, glial scarring around the stimulating electrode will eventually increase electrode impedance and stimulation thresholds.

The conventional transcranial magnetic stimulation (TMS) uses, for the diagnosis and treatment of neurological disorders, hand-held coils positioned over the scalp of the subject to generate very large time-varying magnetic fields (for example, fields with strength exceeding 1 T), which induce currents that modulate neural activity. Experimental evidence suggests that TMS has therapeutic benefits for treating a number of neurological disorders, such as major depression and stroke, for example. However, employing TMS as a standard medical therapy has several limitations. TMS devices must be large to create magnetic fields strong enough to activate neural tissue across large distances. As a result, TMS therapy requires the patient to be in the clinic for long durations, which is both costly and inconvenient. In addition, TMS generally targets superficial cortical regions, because deeper targets (such as the basal ganglia, for example) are simply beyond the range of operational reach of the current technology. Moreover, accurate focus of specific neuronal targets is difficult as spatial control with existing devices is limited. Part of the success of electrical stimulation can be attributed to the array of relatively small electrodes, each capable of independently eliciting activity that is restricted to a focal region of tissue. This begs a question of whether the use of a specifically-dimensioned magnetic device would help to overcome some of the limitations that make TMS inherently unsuitable for chronic prosthetic applications. Currently existing coils smaller than those used in conventional TMS devices can elicit neuronal responses, but such coils are still too large to be surgically implanted. Moreover, currently it is not known whether sub-millimeter-sized or smaller coils can in fact elicit neuronal responses.

Micromagnetic stimulation (μMS) of the tissue with microcoils according to the idea of the invention provides several advantages over conventional electrical stimulation. When turned off, the microcoils are likely to be MRI compatible as long as they are electrically isolated from the adjacent tissue; therefore, the amount of caused heat induction is limited. In addition, microscopic coils may be placed in close proximity to the tissue, thereby improving the spatial control of the elicited neuronal activity. Finally, these coils can be encapsulated with a wide range of biocompatible materials (for example, parylene and liquid crystal polymer), which may help to reduce inflammation.

To demonstrate the utility of such small coils in evoking neural activity, a combination of computational modelling and electrophysiological experiments was used. First, a computational finite element method (FEM) model was created to study the magnetic fields arising from μMS as well as the electric fields they induced. The model suggested that such fields would be sufficient to elicit neuronal activity. This was confirmed by recording from rabbit retinal ganglion cells while stimulating with the small coils and found that μMS does induce neural activity. Both the orientation of the coil and the magnitude of the stimulation parameters influenced the neuronal response, and a wide range of responses could be generated, demonstrating that μMS may provide a suitable alternative to existing electric stimulation devices.

### Electric Field Produced by Embodiments of a Subcortical Microcoil is Sufficient for Neural Activation.

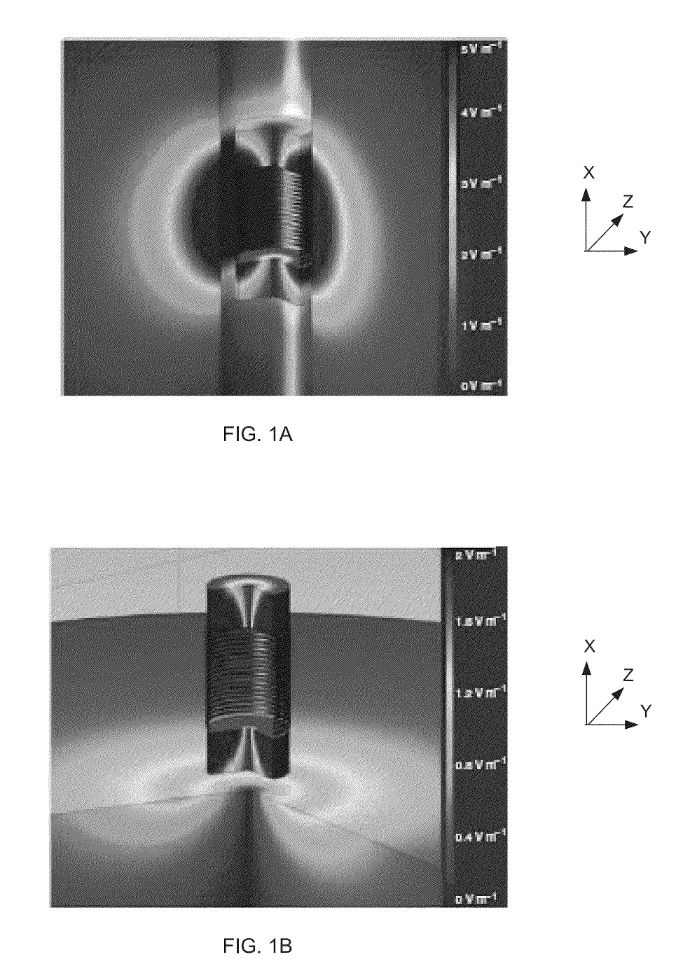

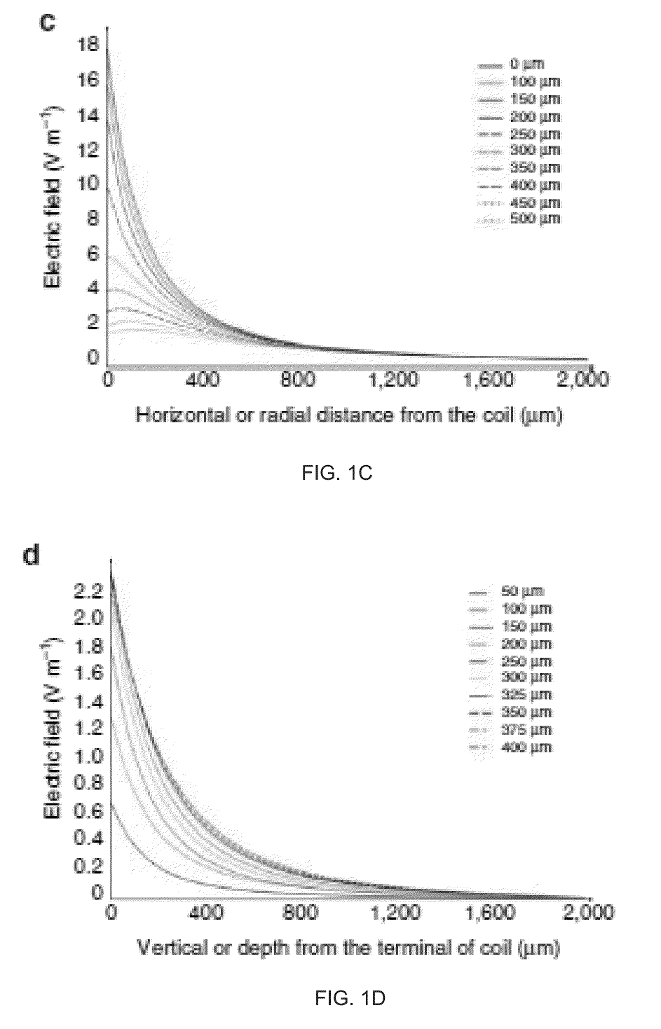

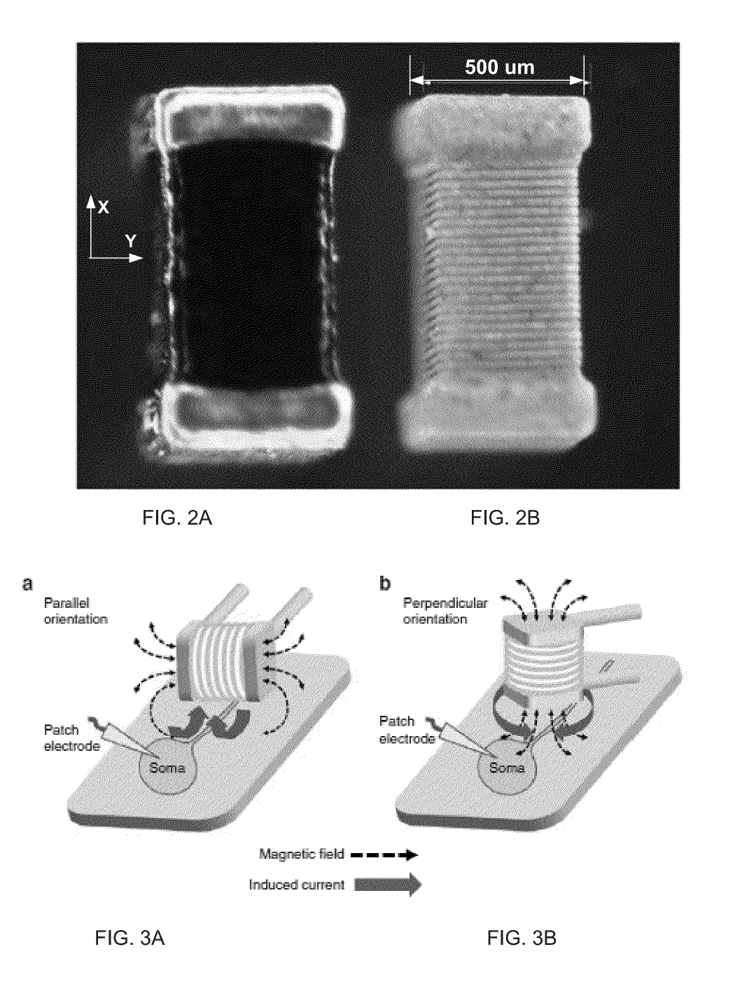

The theoretical equations governing the magnetic and electric fields that arise from current flowing through a coil are well established, but the fields that arise from sub-millimeter-dimensioned (or smaller) coils implanted in biological tissue—and, specifically, their ability to elicit neural activity—have not been previously explored. Magnetic field pulses induce a circulating electric field in the tissue owing to Faraday's law, expressed in one of the four Maxwell equations. Similarly to a boat in a whirlpool, the circulating electric fields (E) generate currents that tend to follow a circular path in conductors and depend on how quickly the magnetic fields (B) generated by the μMS change over time. For the purposes of the present disclosure, the magnitudes of the induced E fields were estimated with the finite-element method (FEM) by solving the set of Maxwell equations simplified by the magnetostatic assumption that all magnetic fields are switching on timescales of microseconds or slower (as in the electrophysiology experiments). The FEM calculations were performed to determine whether microscopic coils used in the electrophysiology experiments could generate E fields large enough to evoke action potentials in neuronal tissue, provided the coils were positioned close to excitable cells. The geometry of one embodiment of the coil was approximated with a cylindrical container of 3 mm radius and 3 mm height, which enclosed different objects: a physiological solution, a quartz core surrounded by the copper solenoid, and top/bottom copper cylindrical metal terminals. The FEM calculations were performed on the model of a cylindrical inductor/coil with was 1 mm in height and 500 micron in internal diameter as well as a 5 μm×10 μm trace section and 21 turns (or coil loops, as shown in FIGS. 1A, 1B), that was positioned inside a uniform volume conductor representing the saline solution or the retinal tissue with similar electrical characteristics. The terminals were two cylinders of 200 μm in radius and 200 μm in height. The quartz core had a 500 μm diameter and was 500 μm in height on top and on the bottom, with copper terminals. Given that the FEM simulations were in cylindrical two-dimensional (2D) coordinates, this cylindrical model approximated the rectangular passive component structure of the actual inductor used in the electrophysiological experiments and shown in FIGS. 2A, 2B. The boundary conditions at the external surfaces of the cylindrical container were set to “magnetic insulation” (i.e., the magnetic field strength was set to zero). The FEM solution provided values of electric (V) and magnetic (A) potentials, which were used to further calculate the E field induced in and around the cylindrical inductor.

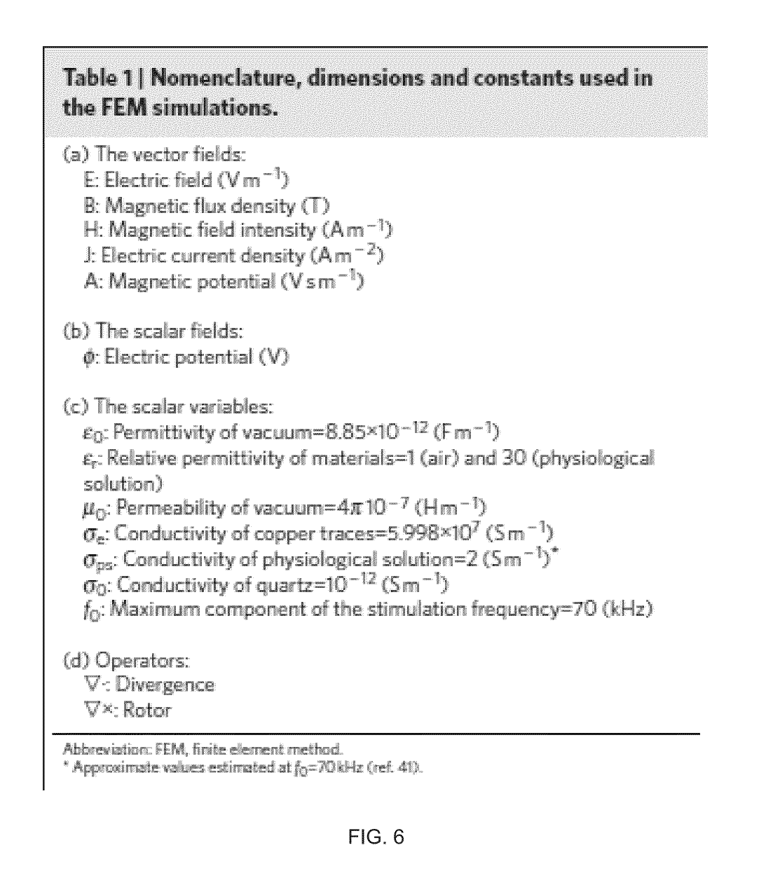

Theoretical Model. All of the electromagnetic quantities introduced in this disclosure are summarized in FIG. 6. An inductor is the ideal magnetic field generator, and it stores the magnetic field energy W generated by the supplied electric current i. Simulations were performed by considering low frequencies, that is, for

f₀²μ₀ε₀l²<<1, (1)

where l is the maximum dimension of the object and f₀ is the maximum current frequency, and ignoring the contribution of the displacement currents (that is, for ∂D/∂t=0). The optimal μMS coil is an inductor characterized by magnetic energy W:

$$W = \frac{1}{2} {\int \int \int }_{Ω} J ( x , y , z ) \cdot A ( x , y , z ) d x d y d z \qquad ( 2 )$$

where A is the magnetic potential (such that B=□×A is the magnetic flux density). In a real inductor, the portion of energy W that is lost is available to elicit neuronal activity even though the loss reduces the Q-factor or the efficiency and the inductance of the coil. Part of energy W in Eq. (2), therefore, is available to elicit neuronal activity. The electric fields and magnetic flux densities were found by solving numerically the following magnetostatic equation

$$\frac{1}{{μ}_{0} {μ}_{r}} ∇ \times ( ∇ \times A ) - σ E = 0 \qquad ( 3 )$$

Here, [symbol] is the electrical conductivity expressed in [S/m]. The induced currents and electric fields in the tissue are expressed by Faraday's law:

$$E = - \frac{\partial A}{\partial t} - ∇ φ \qquad ( 4 )$$

where φ is the scalar potential.

The cylindrical coordinates (r, z, φ) is set, where the microcoil is in the rz-plane (that is, for (r, z₀, φ)) and each turn of the coil has coordinates rᵢ and φ∈[0;2π]. ∇φ is assumed to be zero, because in an unbounded medium the non-zero φ is only due to free charges, and no such sources are present. Furthermore, we considered the frequency domain by assuming time harmonic fields with angular frequency ω and we will perform simulations with the maximum frequency (70 kHz) of the class D amplifier used in the experiments, as the pulse can be represented as ½ of a sinusoidal/cosinusoidal function.

The induced electric fields E=−jωA (from Eq. (4) transformed in frequency domain) were found by solving the following quasi-static equation:

$$( j ω σ - {ω}^{2} {ɛ}_{0} {ɛ}_{r} ) A + ∇ \times ( \frac{1}{{μ}_{0} {μ}_{r}} ∇ \times A ) = {J}^{e} \qquad ( 5 )$$

where J^{e} is the external current, and each turn of the coil, approximated by a circle with radius r and potential Vᵣ, has an electric current amplitude derived from

$$\vert {J}^{e} \vert = \frac{σ {V}_{r}}{2 π r} \qquad ( 6 )$$

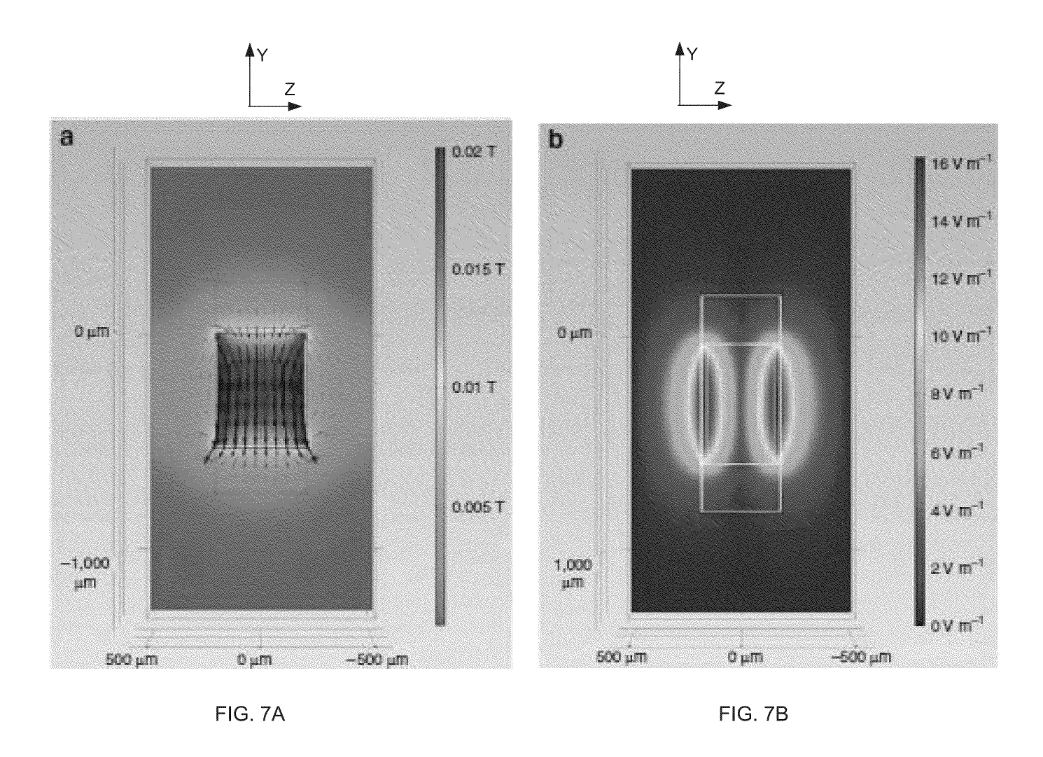

FEM numerical simulations were conducted, based on the above, to study the microscopic magnetic flux density generated by the MEMS microinductor (shown FIGS. 7A, 7B). The FEM simulations were performed in Multiphysics 4.2a with the AC/DC module (COMSOL, Burlington Mass., USA) using the emqa model or the electromagnetic quasi-static approximation.

The solution of the Eq. (5) was sought for the magnetic vector potential A in Eq. (5). There were no weak constraints, and all constraints were ideal. Table 1 of FIG. 6 describes the material properties of the coil and the surrounding physiological solution/tissue and the constant values used in the simulations.

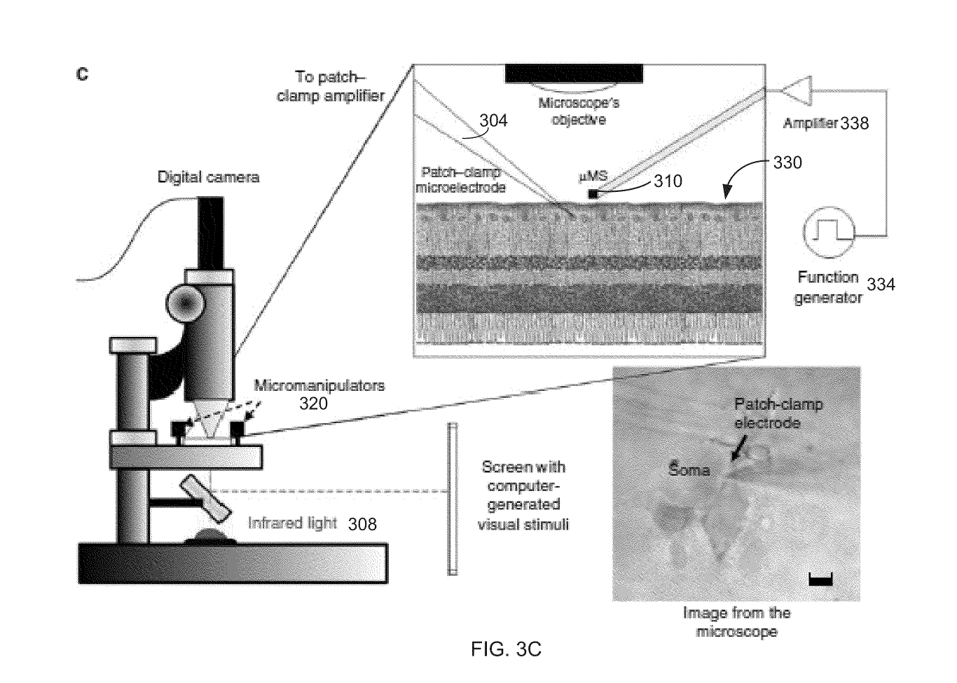

When the long axis of the coil was oriented in parallel to the retinal surface (as schematically illustrated in FIG. 3A), the maximum value of the strength of E was calculated to be about 6 Vm⁻¹ at a distance of 200 μm from the edge of the dielectric (or about 300 μm from the edge of the coil (as shown in FIGS. 1A and 1C). This distance was similar to that used in the electrophysiological experiments discussed below. The magnitudes of the magnetically-induced electric field are comparable to the 10 Vm⁻¹ thresholds for neuronal activation measured by Chan and Nicholson (Chan, C. Y et al., J. Physiol., v. 371, 89-114, 1986).

The strength of induced electric fields was attenuated nonlinearly with increased distance from the coil, FIG. 1C. The maximum values of the induced electric field were much weaker when the coil was oriented with its axis perpendicular to the surface of the tissue at hand (as schematically illustrated in FIG. 3B), and were approximately 1 V m−1 at a distance of 200 μm (300 μm from the edge of the coil), see FIGS. 1B, 1D. Similarly to the parallel orientation, the strength of the electric field decreased nonlinearly with distance when the coil was oriented perpendicular to the retinal surface (FIG. 1D).

In general, the threshold to neural stimulation depends on the so-called strength—duration curve, which is currently not well understood for magnetic stimulation. Therefore, one could, in principle, increase the duration (5 μs) of the induced electric field by extending the duration of the rising or falling current in the μMS coils, as one can only induce an E field by generating a time-varying B field in the μMS coils. Such changes are limited, however, by the peak current of about +/−10 A before the microcoil is damaged. According to collected empirical data, the copper traces used in present embodiments of the microcoil may carry a 6.6×10⁹ Am⁻² DC-current density for 5 . . . 6 μs pulses before melting occurs, a value that is in line with the maximum current density used in TMS (Fried, S. I. et al., J. Neurophysiol., v. 101, 1972-1987, 2009). Alternatively or in addition, one could increase the strength of the induced E field by increasing the slope of the current pulse in the coils, but this would reduce the pulse length because of the limitation imposed by the maximum current in the coil as discussed above.

Experimental Verification of Neural Activation with Micro-MS for Varying Spatial Orientations of Microcoils.

To experimentally confirm that neurons could in fact be activated by μMS process of the invention, a series of electrophysiological experiments was conducted that measured the response of retinal ganglion cells to stimulation from a small, commercially available magnetic coil (shown in FIGS. 2A, 2B) that was assembled into a custom μMS device of the invention. To the best of knowledge of the inventors, related art is silent about evaluation of the activation of neuronal tissue with the use of coils that are specifically dimensioned to be implanted subcortically.

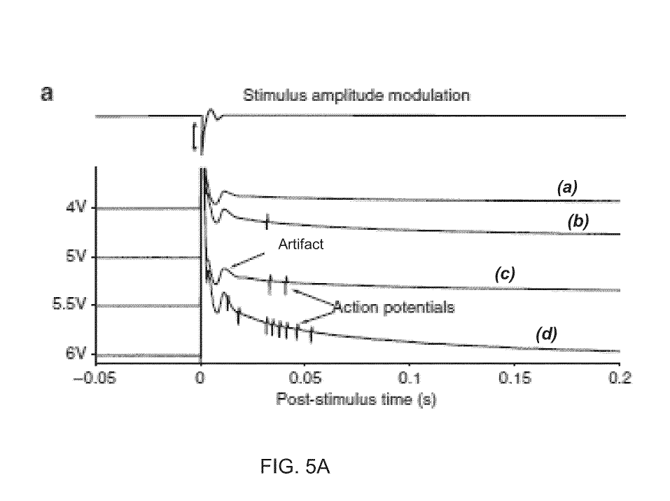

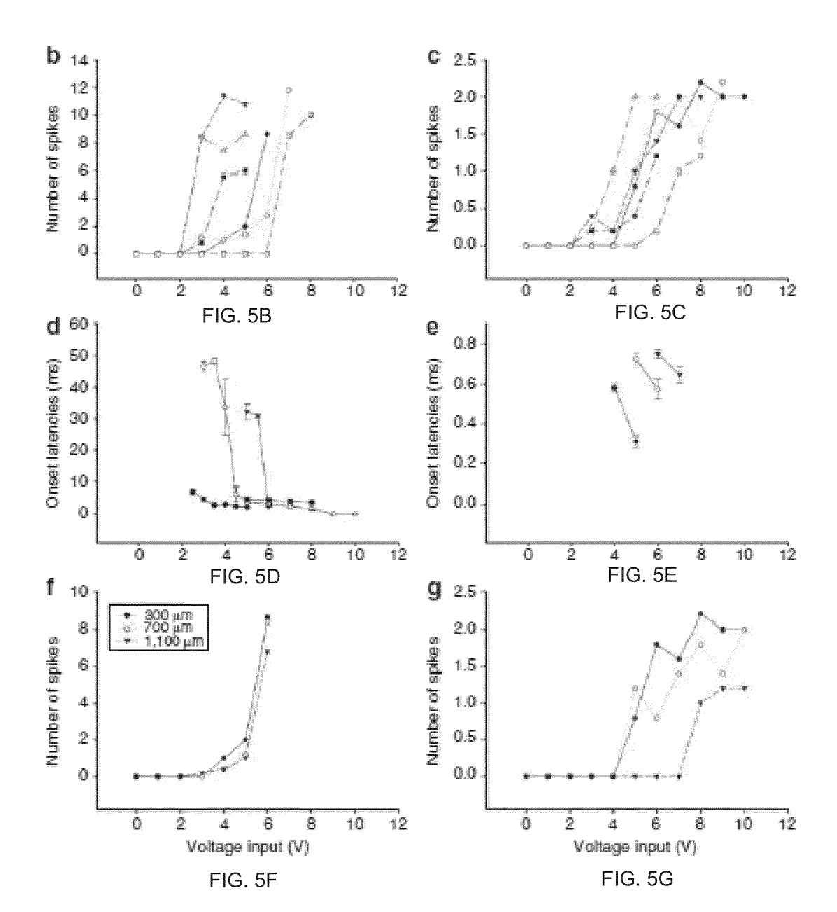

Set-Up: Microcoils and Tissue: Assembled microcoils were manually coated with a xylene-based dielectric varnish (Gardner Bender, Milwaukee, Wis., USA). This operation resulted in a non-uniform coating of dielectric as well as an irregular (non-smooth) outer surface. As the dielectric was opaque to the infrared illumination system used during in vitro experiments, it was necessary to establish the approximate location of the coil relative to the outer boundaries of the dielectric-coated assembly (typically ˜200 μm). The determination of the height of the dielectric-coated coil in the assembly above the surface of the tissue during an experiment was addressed using preliminary measurements under bright illumination revealed the position of the coil within the dielectric and were also used to determine the exact outer limits for each (coated) coil assembly. In this manner, the bottom edge of the coil assembly was determined relative to a focal point at or near the top surface of the assembly, and the height of the coil above the retinal surface could be reasonably estimated. In this manner, the distance from the surface of the tissue to the closest edge of the coil could be reliably controlled and was set to 100 μm in most experiments. The approximated thickness of the dielectric was 200 μm and, therefore, the electrical-trace-to-retina distance was taken as 300 μm. The actual variability of the thickness of dielectric was estimated to be +/−50 μm. The increases in distance associated with the experiments (discussed below in reference to FIGS. 5A through 5G) were obtained via a 400 (or 800) μm translation in the z-direction using the micromanipulator. The μMS coil assemblies were tested before and after each experiment to ensure that there was no leakage of current. If present, such currents could have produced the observed neural activity. The coils were submerged in physiological solution 0.9% NaCl) and the impedance between one of the coil terminals and an electrode immersed the physiological solution was measured before and after each electrophysiological experiment. Impedances above 5 MΩ were considered indicative of adequate insulation. Liquid Teflon® was selected because of its high dielectric strength (>100 Vμm⁻¹), and the 5 MΩ value was based on the fact that Teflon has very low conductivity (typically 10 to 23 Sm⁻¹) and that the tester was not able to reliably measure high impedances as it had a low dynamic range.

The extraction and mounting of the target tissue as well as the use of cell-attached patch clamp recordings followed well-established procedures. Patch pipettes were used to make small holes in the inner limiting membrane, and ganglion cells with large somata were targeted under visual control, as shown in FIG. 3C.

In reference to FIG. 3C, to measure responses cells, the extracted retina was positioned ganglion cell side up in a small chamber (˜1 ml volume) and perfused with oxygenated Ames medium. The responses to the stimuli were recorded with a cell-attached patch electrode 304 (4-8 MΩ) positioned on the surface of the soma of targeted ganglion cells (which is referred to as a cell-attached configuration) to detect action potentials elicited in response to magnetic stimulation. Before the onset of μMS testing, responses to full-field flashes of light 308 were measured to ensure viability of each targeted ganglion cell; only those cells that generated robust light responses were used for subsequent μMS stimulation. All cells that generated spiking in response to light stimuli also generated spikes in response to μMS. The experimental results discussed below are derived from recordings in 12 ganglion cells (6 different retinas).

The μMS coil assembly 310 was fixed in the micromanipulator(s) 320 such that the main axis of the coil 310 was oriented either parallel or perpendicular to the retinal surface 330, as shown schematically in FIGS. 3A and 3B. The coil assembly was lowered into the bath until its bottom edge was 100 μm above the surface of the tissue; this corresponded to a coil to tissue separation of about 300 μm. Five repeats were performed for each parameter set, that is, every time a parameter was changed. The coils were coated with a biocompatible high-strength insulator, and impedance was tested at the beginning and end of each experiment to eliminate the possibility that responses were mediated by a leakage of current from the wire into the retinal preparation.

Set-Up: Micro-MS Drive. The output of a function generator (AFG3021B, Tektronix, Beaverton, Oreg., USA) was connected to a 1,000 W audio amplifier 338 (PB717X, Pyramid, Brooklyn, N.Y., USA) with a bandwidth of 70 kHz. Positive and negative pulses were created alternately by the function generator at a rate of 1 pulse per second. Pulse amplitudes ranged from 0 V to 10 V in steps of 0.5 V and the rate of increase of the leading edge was 18 ns/V; the decrease of the trailing edge occurred at an equal rate. The output of the amplifier 338 included a sharp peak followed by a damped cosine waveform (monitored with a DPO3012 oscilloscope; Tektronix, Beaverton, Oreg.). The peak had maximum amplitudes ranging from 0 V to 46 V with leading/trailing edge slopes of 80 ns/V. The duration of the peak was approximately 20 μs. The amplitude of the damped sinusoid was smaller than that of the peak and ranged from 0 V to about 12 V; the duration of the damped sinusoid was about 12 ms.

Data Processing. Raw waveforms were recorded at a sample rate of 20 kHz and processed with custom software written in MATLAB. Each elicited waveform contained an electrical artifact arising from the μMS pulse; the artifact lasted approximately 20 ms and was nearly identical for trials with identical stimulus conditions. Many elicited responses also contained a series of action potentials (spikes); these were confirmed as spikes by comparing them to those spikes elicited in response to light stimuli. The timing of individual spikes was determined with a “matched filter”—the average light-elicited spike was cross-correlated with the response waveform; peaks in the cross correlation were used to assign timing of individual action potentials.

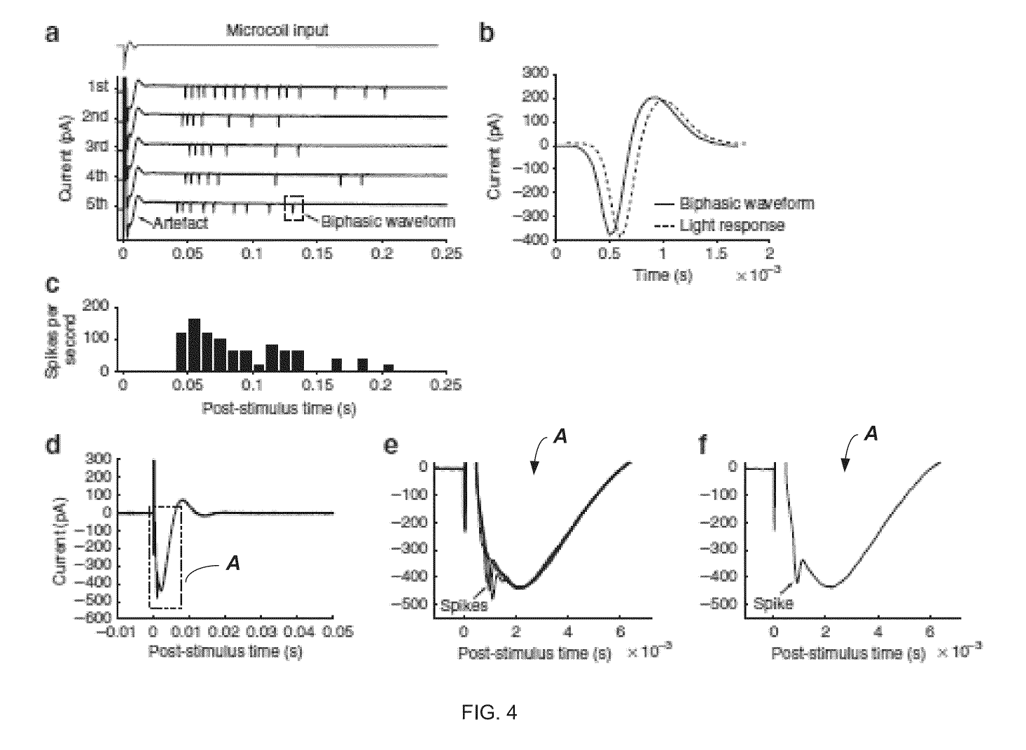

Action potentials formed in response to μMS stimulation according to the idea of the invention were consistent with the results of theoretical calculations discussed above, FIGS. 4A, 4B, 4C, 4D, 4E, 4F. With the axis of the coil oriented parallel to the retinal surface, a single μMS pulse was shown to elicit complex responses that included a prolonged stimulus artifact (as shown in FIG. 4A, duration of ˜20 ms) followed by a series of biphasic waveforms. The amplitude and kinetics of individual biphasic waveforms were nearly identical to that of action potentials elicited in response to light stimuli (FIG. 4B), strongly suggesting that the biphasic waveforms were in fact action potentials. There were slight variations in the number and/or latency of elicited spikes from trial to trial (FIG. 4A, each row is a separate trial from the same cell), although the general features of the response were consistent across trials (FIG. 4C). Similar responses were seen in all six cells for which the coil was oriented parallel to the retinal surface.

In contrast to the burst responses elicited when the orientation of the coil was parallel to the surface of the tissue, the response to μMS according to the idea of the invention included only one or two spikes when the coil orientation was perpendicular to the surface (as shown in FIGS. 4D, 4E, and 4F). The first spike always occurred within the stimulus artifact and its latency ranged from about 0.3 to about 0.6 ms. Similar responses were observed in all 6 cells for which the coil was oriented perpendicularly to the surface of the tissue at hand.

Changes to the amplitude of the μMS pulse altered the response to stimulation (FIGS. 5A, 5B, 5C, 5D, 5E, 5F, and 5G). In the parallel configuration, increases in the amplitude of the pulse elicited a larger number of spikes (see FIG. 5A, showing responses to stimuli with amplitudes of 4 V versus 6 V). To examine the sensitivity to amplitude changes across the population of cells, the number of spikes elicited at each amplitude was averaged across five trials (FIG. 5B); average counts were then plotted as a function of stimulus amplitude. In shown plots, lines connect individual points from the same cell. All cells tested in the parallel configuration (n=6) exhibited an increase in the level of spiking with increased amplitude of stimulation, FIG. 5B. Similarly, the responses arising from the stimulation in a perpendicular orientation were also sensitive to stimulus amplitude, FIG. 5C (n=6). However, as there were only 1 or 2 spikes in a response curve elicited in this orientation, the increase in sensitivity was observed as an increased likelihood of eliciting the spike (or doublet). Increases in stimulus amplitude were also found to reduce the latency of elicited spikes (FIGS. 5D, 5E). This reduction in latency occurred in both spatial configurations (parallel: n=5/5; perpendicular: n=3/3). For the stimulation with the perpendicular configuration, in which latencies were small to begin with, the decreases in latency were correspondingly small as well. In the parallel configuration, a small decrease in latency was observed in all cells tested, but for two cells, the drop was >25 ms. While the reason for the large variability in latency shifts is not particularly clear at the moment, it is possible that alternative, faster-acting mechanisms become activated at higher amplitude levels of stimulation.

For each spatial configuration between the microcoil and the surface of the tissue, the sensitivity to stimulation was explored as the distance between the μMS coil and the targeted cell was varied. Similar to the experiments discussed above, the number of spikes in a response curve elicited by signals with different amplitudes was determined for a fixed separation between coil and cell. The separation was then increased from 300 to 700 μm and then to 1,100 μm; at each predetermined separation, a new series of measurements was carried out. Responses to stimulation from both parallel and perpendicular orientations are shown in FIGS. 5F and 5G, respectively. A slight decrease in sensitivity was observed as the coil was moved away from the cell for all 4 cells in which this experiment was conducted (n=4); however, robust responses were observed even for cell to microcoil separations of 1,100 μm. These results suggest that the μMS method of the invention may create a fairly large zone of activation. Because the rate at which threshold increases for electric stimulation is proportional to the square of the distance between the cell and the electrode, these results also suggest that the sensitivity to distance is less for μMS than that of electric stimulation of the related art.

At high stimulus amplitudes, the μMS pulse generated a ‘disturbance’ in the perfusion bath, which appeared as a transient flow of perfusion solution across the video monitor in which the cell and surrounding environment were observed (in a set-up discussed in reference to FIG. 3C). Such transient flow is not likely to be needed for neuronal activation to occur as there was no observed disturbance at lower stimulus amplitudes, even though spiking in the response curves was elicited. Further, the latency of the first spike was <1 ms in one-half of the cells we tested (n=6), strongly suggesting that activation was non-synaptic (that is, direct) in these cells. Therefore, even if a distal disturbance, outside the field view of the camera, activated neurons presynaptic to the ganglion cell, the delays associated with synaptic transmission would likely result in onset latency (for ganglion cell spiking) of greater than 1 ms. The increased flow did not alter the position of the cell, the position of the recording electrode, or the impedance of the patch connection between the two, even at high amplitudes. This is the argument against the possibility that responses arose from some form of mechanotransduction (in other words, the movement of the patch electrode did not act as a mechanical stimulus). However, the possibility that, at high amplitudes, the transient change in flow rate influenced the responses cannot be ruled out at the moment. Although the experiments did not include an investigation of the source of this disturbance, it seems likely to arise from some form of mechanical resonance in the coil.

The experiments conducted in this study represent an important first step in exploring the potential clinical applications of μMS. Computational simulations predicted, as shown, that the coil design and range of stimulation parameters used here would generate, subcortically, an electric field that was sufficiently strong to activate neurons. Consistent with these findings, a series of electrophysiological experiments revealed that neural activity is elicited in response to μMS. Potential contributions from several non-magnetic factors including leaking electrical current, heating of tissue, and mechanical vibration were all eliminated allowing us to conclude that small magnetic fields can elicit action potentials. As embodiments of the coils are small enough to be implanted into both the cortex and deep subcortical nuclei, the findings presented in this disclosure raise the possibility that μMS may be a viable alternative to existing DBS devices and other neural prosthetics.

Previous studies of the retinal ganglion cell response to electric stimulation revealed two modes of activation: (1) direct activation of the ganglion cell and (2) indirect, or secondary activation resulting from activation of neurons presynaptic to the ganglion cell (see, for example, Loewenstein, J. I. et al., in Arch. Ophthalmol., v. 122, 587-596, 2004; or Fried, S. I. et al, in J. Neurophysiol., v. 95, 970-978, 2006). Each mode of activation has a distinct response signature: direct activation typically results in a single action potential with latency ≦1 ms, while the indirect response is more complex and typically consists of one or more bursts of spiking that have slower onset and persist for tens or hundreds of ms.

The results of this research unexpectedly showed that both modes of activation are also elicited according to the μMS method of neuronal stimulation of the invention. Specifically, those neurons in close proximity to the (circular) end surfaces of the coil elicited one or two spikes only, while those neurons along the cylindrical lengths of the coil exhibited bursts of spikes. Therefore, it has been demonstrated that micro-magnetic stimulation can elicit neuronal responses through at least two different modes of activation. Moreover, it is likely that the mode of activation is dependent on the location of the cell relative to the geometry of the coil: those ganglion cells close to the circular end surfaces of the coil were activated directly whereas those ganglion cells closer to the cylindrical lengths of the coil were activated secondary to activation of deeper (presynaptic) neurons. The mechanisms underlying activation are thought to be different for the two modes of activation with the use of the present invention. Direct activation is thought to occur through rapid depolarization of the voltage-gated sodium channels in the proximal and distal portions of the axon. The mechanism underlying indirect activation is not known, but modelling studies suggest that as the slower acting voltage-gated calcium channels in the axon terminal become activated, the ensuing inflow of calcium mediates an increased release of synaptic neurotransmitter. The temporal kinetics of the induced electric field from μMS according to an embodiment of the invention was presumably the same at all locations and therefore it is somewhat surprising that responses to a given pulse were different for different locations around the coil. One possibility is that other mechanisms (that is, not ion channel kinetics) contribute to the response differences at different regions. Differences in the spatial properties of the induced electric field at the two locations (FIGS. 1A, 1B) may have a role, although further work will be needed to determine the actual mechanism(s).

Regardless of the exact mechanism of activation, the ability of the micro-MS procedure according to the invention to generate both modes of activation may serve to enhance clinical outcomes. For example, methods that can selectively target presynaptic terminals may help to maximize the effectiveness of DBS stimulation. In addition, the orientation of the μMS coil could be used to mitigate the side effects arising from unwanted axonal activation, that is, a primary side-effect of DBS therapy is the activation of adjacent tissue that result in the paresthesias. Further enhancements to selectivity of neural activation could arise from changes to the coil design, for example, lengthening the coil and reducing the diameter might help to further avoid the activation of axons. The responses to μMS exhibited similarities to the responses elicited by conventionally-used electric stimulation. For example, a larger number of spikes were elicited as the amplitude of the μMS pulse increased. In addition, the number of elicited spikes decreased as the distance between coil and cell increased, although the decrease in sensitivity for μMS was smaller than that for electric stimulation. These findings suggest that the volume of activated neurons arising from a μMS coil could be considerably larger than that from conventional electrical stimulation devices. This may prove to be beneficial because chronic electric stimulation technologies lose efficacy with the formation of glial scars and μMS activation may still be effective even for the largest size scars. The performed simulations suggest that sensitivity to distance can be modulated by changes in the coil geometry (and/or the parameters of stimulation) and further testing will be needed to determine how well the region of activation can be tailored to the needs of specific applications.

Although the current study is prefaced on the potential clinical applications for the μMS coils, there is also a potential for this technology to be utilized in experimental settings. Specifically, because the proposed technological modality is inherently MRI compatible, it could be used as an alternative to TMS, FES, or peripheral electrical stimulation during imaging studies. In addition, because μMS coils can be used in both in vivo and in vitro preparations, there are opportunities to use these devices to investigate the mechanism of action of DBS, FES, or TMS. Finally, although we have demonstrated computationally and empirically that μMS can modulate neuronal activities, further studies are needed to understand the relationship between the parameters of magnetic stimulation and neuronal activation as well as the effects on μMS on different neuronal elements (for example, presynaptic, postsynaptic, soma and axonal) and the spatial characteristics of μMS fields in different brain tissues.

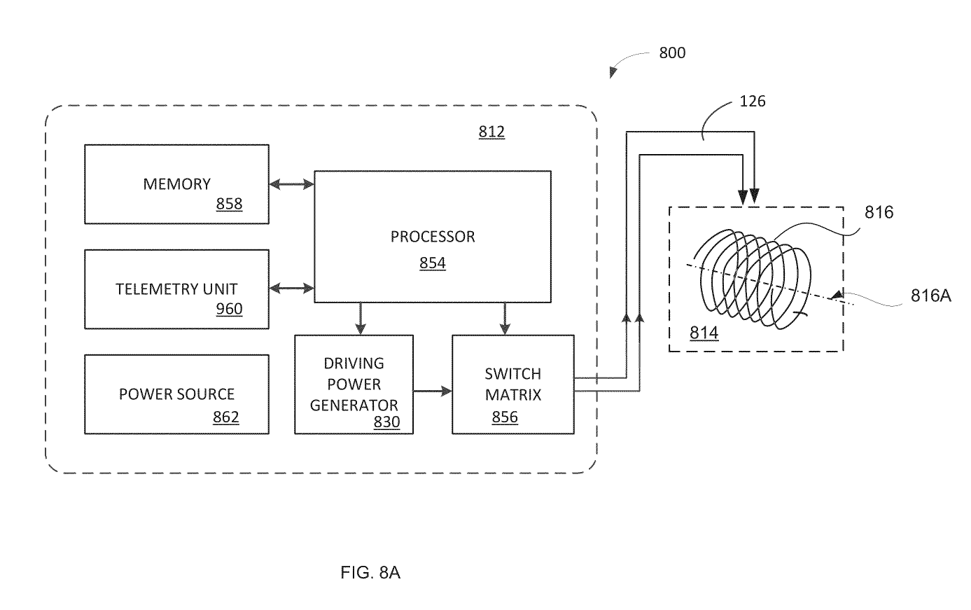

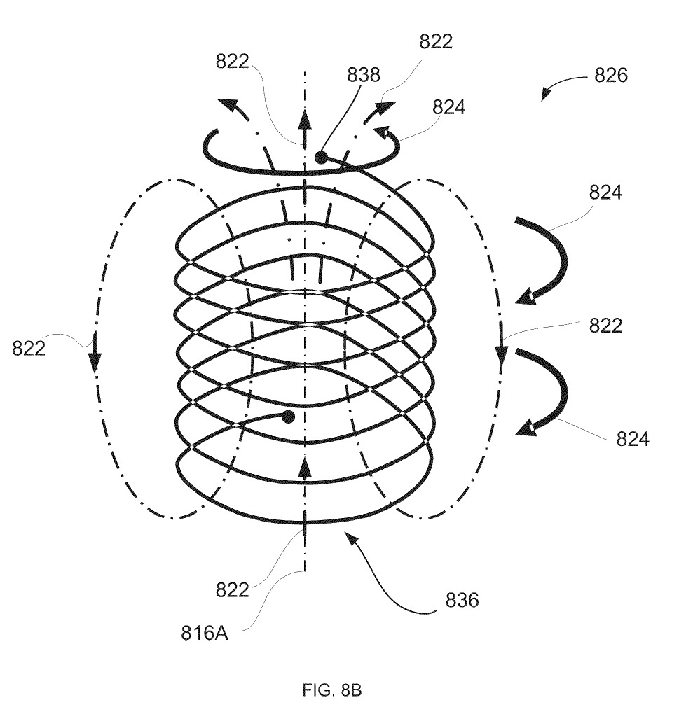

Example of a System. An example of a subcortical tissue stimulation system 800, shown in FIG. 8A, includes a stimulator 812 coupled to a biocompatible device 814 (which may be structured as an implantable device, without reference to any particular structure) that contains an electromagnetic microcoil 816 (having a respectively corresponding axis 816A) that is disposed inside the device 114 and is fluidly isolated from the ambient surrounding the device 814. The stimulator 812 includes a drive power generator 830 that generates electrical pulses for delivery to a targeted stimulation site in the neuronal tissue 818 via the device 814 as a result of installation of the device 814 into the tissue. The applied electrical pulses cause a given microcoil 816 to produce magnetic field with a characteristic spatial distribution indicated, with traces 822, in FIG. 8B. For example, a magnetic field vector in the middle of the coil is directed substantially co-linearly to the microcoil axis. The magnetic field, in turn, induces electrical currents (indicated with traces 824). In a specific implementation, the device 814 together with a subcortical target neuronal tissue in the vicinity of which it is disposed, may be placed in a conventional MRI system (not shown) to perform magnetic-resonance imaging of the tissue in conjunction with the process of eliciting neuronal responses as discussed above.

In the simplest implementation, as in further reference to FIGS. 8A and 8B, a microcoil 816 may be shaped as three-dimensional spiral 836 including loops of a metallic wire and having electrical terminals 838. The space between at least some of the individual loops may be optionally and at least partially filled with a dielectric material (not shown). Alternatively or in addition, a microcoil 816 may be overcoated with a dielectric material such as, for example polytetrafluoroethylene (PTFE; Teflon), polyurethane, polyimide, parylene or liquid crystal polymers (LCPs). These biocompatible materials can be used for coating of the microcoil 816 as well as to construct the body of the biocompatible shaft of the device 814. In the embodiment in which a given microcoil is electrically connected to the stimulator 812 with via an electrical lead 826, the device 814 also includes such electrical conducting lead(s) or member(s) that are connected to the terminals 838 (shown in FIG. 8B).

Referring again to FIG. 8A, the stimulation system 812 may further include a processor 854 to set the parameters of driving electrical pulses applied to the subcortically placed device 814. The processor 854 may be realized by one or more microprocessors, digital signal processors (DSPs), Application-Specific Integrated Circuits (ASIC), Field-Programmable Gate Arrays (FPGA), or other equivalent integrated or discrete logic circuitry. The stimulation system 800 may further include (optionally) a switch matrix 856 to apply the stimulation pulses across selected microcoil 816 within a single portion of the implant 814 or within two or more implant portions. The stimulation pulses may be applied in a bipolar or multipolar arrangement, in which multiple microcoils 816 (within the same device 814) are selected for delivery of stimulation pulses, for example, across or among different microcoil pairs or groups. Alternatively, the stimulator 812 may include multiple pulse generators 830, each coupled to and controlling given microcoil(s) 816.

A tangible non-transitory computer-readable memory 858 may be provided to store instructions for execution by the processor 854 to control the pulse generator 833 and the switch matrix 856. For example, the memory 858 may be used to store programs defining different sets of stimulation parameters and microcoil combinations. Other information relating to operation of the stimulator 812 may also be stored. The memory 858 may include any form of computer-readable media such as random access memory (RAM), read only memory (ROM), electronically programmable memory (EPROM or EEPROM), flash memory, or any combination thereof.

A telemetry unit 860 supporting wireless communication between the stimulator 812 and an external programmer and/or display device (not shown) may be provided. The processor 854 controls the telemetry unit 860 to receive programming information and send operational information. Programming information may be received from an external clinician programmer or an external patient programmer. The wireless telemetry unit 860 may receive and send information via radio frequency (RF) communication. The display device may be configured to form a visually-perceivable representation of the results of interaction between the field(s) generated by the microcoil systems of the implant 814 and the target neural tissue.

A power source 862 delivers operating power to the components of the stimulator 812 including the microcoil(s) 816. The power source 862 may include a rechargeable or non-rechargeable battery or a power generation circuit to produce the operating power. In some embodiments, battery recharging may be accomplished through proximal inductive interaction between an external charger and an inductive charging coil within the stimulator 812. In other embodiments, operating power may be derived by transcutaneous inductive power generation, e.g., without a battery.

In a related embodiment, the processor 854 is specifically programmed to govern the operation of the stimulator 812 to cause the amplitude and/or frequency modulation of the magnetic field(s) generated by at least one of the microcoil(s) 816.

For the purposes of this disclosure and appended claims, the use of the term “substantially” as applied to a specified characteristic or quality descriptor means “mostly”, “mainly”, “largely but not necessarily wholly the same” such as to reasonably denote language of approximation and describe the specified characteristic or descriptor so that its scope would be understood by a person of ordinary skill in the art. The use of this term both in the present disclosure and the appended claims neither implies nor provides any basis for indefiniteness and for adding a numerical limitation to the specified characteristic or descriptor. For example, a reference to a vector or line being substantially parallel to a reference line or plane is to be construed as such vector or line extending along a direction that is the same as or very close to that of the reference line or plane (for example, with angular deviations from the reference direction that are considered to be practically typical in the art). As another example, the use of the term “substantially flat” in reference to the specified surface implies that such surface may possess a degree of non-flatness and/or roughness that is sized and expressed as commonly understood in the art in the specific situation at hand.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, the following disclosure may describe features of the invention with reference to corresponding drawings, in which like numbers represent the same or similar elements wherever possible. In the drawings, the depicted structural elements are generally not to scale, and certain components may be enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed.

The invention as recited in claims appended to this disclosure is intended to be assessed in light of the disclosure as a whole.

While the invention is illustrated in reference to some specific above-described examples of embodiments, most of which illustrate the application of the coil implant of the invention to intraocular tissue and stimulation of such intraocular, retinal tissue with such implanted coil, it will be understood by those of ordinary skill in the art that modifications to, and variations of, the illustrated embodiments are easily made without departing from the disclosed inventive concepts. In particular and specifically, embodiments of the coils of the invention and method of operation thereof as applied to sub-cortical neural tissue adjacently to which such embodiments are implanted are within the scope of the invention. The dimensions of the coils and methods of their operation remain substantially the same regardless of what particular neural tissue is chosen to be a target tissue. Similarly, while the description of electrical stimulus applied to the terminals of a microcoil of the invention was discussed in reference to an electric pulse, different waveforms of the stimulus (such as, for example, a sinusoidal or trapezoidal waveform) can be used in a related embodiment. Disclosed aspects, or portions of these aspects, may be combined in ways not listed above. Accordingly, the invention should not be viewed as being limited to the disclosed embodiment(s).

What is claimed is:

1. A method for stimulating a target tissue with a microcoil implanted therein, the method comprising:
applying an electrical stimulus to terminals of the microcoil positioned in the vicinity of said target tissue to generate a first magnetic field at said tissue,
wherein said first magnetic field has substantially the same strength at said subcortical tissue as a strength of a second magnetic field,
wherein the second magnetic field is defined inside tissue during a transcranial magnetic stimulation procedure,
wherein said implanted microcoil has dimensions of two millimeters or less;
eliciting a response of said target tissue with said first magnetic field, said response having a latency; and
modulating a response of said target tissue based on defining a spatial orientation of said microcoil with respect to a surface of said tissue.

2. A method according to claim 1, further comprising positioning of said implantable microcoil at a sub-millimeter distance from a surface of said target tissue.

3. A method according to claim 1, further comprising eliciting a response, from said target tissue, to light illuminating said tissue.

4. A method according to claim 1, further comprising reducing said latency by increasing an amplitude of said electric stimulus.

5. A method according to claim 1, wherein said eliciting a response includes at least one of (i) a direct activation of a cell of the target tissue with said first magnetic field and (ii) an indirect activation of said cell resulting from activation of neurons presynaptic to the said cell.

6. A method according to claim 1, wherein said eliciting a response includes eliciting a response, to said first magnetic field, of said target tissue during a procedure of magnetic resonance imaging (MRI) of said target subcortical tissue.

7. A method according to claim 1, wherein said target tissue includes subcortical tissue.

8. A tissue stimulator system comprising:
a biocompatible unit including a magnetic coil that is structured
to be implanted in the vicinity of a target tissue and
to generate a first magnetic field in a target tissue in response to an electrical stimulus applied to said coil,
wherein said first magnetic field has substantially the same strength as a second magnetic field,
wherein the second magnetic field is defined in said target tissue during a transcranial magnetic stimulation procedure;
a stimulator operably coupled to said biocompatible unit and containing a power drive providing an electric pulse to said magnetic coil; and
a processor configured to govern parameters of said electrical stimulus.

9. A system according to claim 8, wherein said implanted coil has sub-millimeter directions and is implanted, in operation, at a sub-millimeter distance from a surface of the target tissue.

10. A system according to claim 8, wherein said target tissue includes subcortical tissue, and further comprising a magnetic resonance imaging (MRI) system configured to image the target subcortical tissue to which an input has been applied by said tissue stimulator system.

11. A system according to claim 8, wherein said processor is programmed to change, in operation of said system, a latency of response of said target tissue to said electrical stimulus.

12. A system according to claim 8, wherein said coil is disposed in association with the biocompatible unit such as to elicit, in operation of said system, a response from the target tissue that includes at least one of (i) a direct activation of a cell of the target tissue with said first magnetic field and (ii) an indirect activation of said cell resulting from activation of neurons presynaptic to said cell.
